(19) United States
(12) Patent Application Publication
(10) Pub. No.: US 2009/0118597 A1
(43) Pub. Date: May 7, 2009

(54) Neural Signal Processing

(76) Inventors: Perry A. Mills, Arden Hills, MN (US); Marina V. Brockway, Shoreview, MN (US); Brian P. Brockway, Shoreview, MN (US)

Correspondence Address: FISH & RICHARDSON P.C., P.O. BOX 1022, MINNEAPOLIS, MN, 55440-1022, US

(21) Appl. No.: 12/265,541

(22) Filed: Nov. 5, 2008

Related U.S. Application Data
(60) Provisional application No. 60/985,562, filed on Nov. 5, 2007.

(51) Int. Cl. A61B 5/04

(52) U.S. Cl. 600/301; 600/300

(57) ABSTRACT

A method of monitoring neural signal activity with an implantable monitoring device implanted in a living subject includes amplifying a neural signal having a signal bandwidth of at least 1000 Hz. The method also includes processing the amplified neural signal to produce a reduced-bandwidth derivative neural signal representative of gross activity of the neural signal, and wirelessly transmitting the derivative neural signal from the implantable monitoring device for receipt by a receiver external of the living subject.

### CROSS-REFERENCE TO RELATED APPLICATIONS

This application claims priority from U.S. Provisional Patent Application Ser. No. 60/985,562, filed on Nov. 5, 2007, and entitled “Neural Signal Processing,” the contents of which are hereby incorporated in their entirety by reference.

### TECHNICAL FIELD

This disclosure relates to acquiring, processing, and telemetering physiological signals, and more particularly to acquiring, processing, and telemetering neural signals.

### BACKGROUND

Implantable devices have been used to monitor vital physiological signals in humans and animals. For example, implantable devices have been used to measure body temperature or pressure (e.g., arterial pressure, venous pressure, intra-cranial pressure) for monitoring a human patient, or for collecting data for biomedical research using a laboratory animal. Implantable devices have also been used to measure electrocardiogram (ECG) signals, electroencephalogram (EEG) signals, and electromyogram (EMG) signals. A physician or researcher can use information present in the measured signals to assess the condition of the implanted subject and make decisions on how to proceed with future treatment or analysis, or to assess an administered therapy's effect on the research subject.

Neural signals, which are produced by the firing of neurons and which code information related to bodily functions in humans and animals, may also be measured. In some cases, neural signal activity may precede other physiological changes in the body. Researchers may be interested in monitoring neural signal activity of a research subject, such as a laboratory animal, to note changes in activity that may warn of a physiological change that will later manifest.

Measuring neural signals using an implantable device can be challenging because of the high bandwidth of the neural signals to be measured, as compared to the relatively lower bandwidth of the vital signals or certain electrical signals within the subject. For example, neural signal activity may have a bandwidth in the range of about 1 KHz to about 3 KHz, whereas bandwidths of other signals of interest may be much lower, such as below about 250 Hz in the case of many vital or electrical signals (e.g., temperature, blood pressure, ECG, etc.). Because of this high bandwidth of the neural signals to be measured, sampling to acquire the signals, processing the signals, and especially telemetering the signals from the implantable device to an external receiving device can consume large amounts of current, which may result in rapid battery depletion for the implantable device. As a result, batteries of such implantable devices may need to be frequently replaced or recharged, which can be inconvenient for several reasons. For example, in a laboratory setting, replacing or recharging the battery of an implanted device may involve surgery or handling of the animal, which may stress the animal and affect future readings. Also, data may not be collected using the implanted device during battery replacement or recharging, or for a period of time following the replacement or recharging.

### SUMMARY

Disclosed herein are devices, methods, and systems that can be used for acquiring a high-bandwidth neural signal and deriving a reduced-bandwidth signal that retains sufficient information from the high-bandwidth signal to permit research assessments of neural activity.

In a first general aspect, a method of monitoring neural signal activity with an implantable monitoring device implanted in a living subject includes amplifying a neural signal having a signal bandwidth of at least 1000 Hz. The method also includes processing the amplified neural signal to produce a reduced-bandwidth derivative neural signal representative of gross activity of the neural signal, and wirelessly transmitting the derivative neural signal from the implantable monitoring device for receipt by a receiver external of the living subject.

In various implementations, the derivative neural signal may have a signal bandwidth of less than about 250 Hz. The method may also include sampling the amplified neural signal and wirelessly transmitting the sampled signal for receipt by a receiver external of the living subject, wherein the sampling preserves the full bandwidth of the amplified neural signal. Transmission of the derivative neural signal and the full-bandwidth sampled signal may occur simultaneously. Processing the amplified neural signal may include rectifying and integrating the amplified neural signal or detecting successive local extrema of the amplified neural signal. The neural signal may be accessed using electrodes in contact with a renal nerve of the living subject. The method may also include assessing the derivative neural signal to predict occurrence of a future physiological change for the living subject. The method may also include using the derivative neural signal to modulate a periodic waveform. The method may also include determining and subtracting an offset from the derivative neural signal, where the offset represents a contribution of noise to the derivative neural signal.

In a second general aspect, an implantable monitoring device includes an acquisition circuit configured to amplify a neural signal having a signal bandwidth of at least 1000 Hz. The device also includes a processing circuit configured to process the amplified neural signal to produce a derivative neural signal representative of gross activity of the neural signal. The device further includes a transmitter circuit configured to wirelessly transmit the derivative neural signal from the implantable monitoring device for receipt by a receiver external of the living subject.

In various implementations, the device may further include a control circuit that when activated causes the transmitter circuit to wirelessly transmit the amplified neural signal for receipt by a receiver external of the living subject. The device may further include a second transmitter circuit, and transmission of the derivative neural signal and the amplified neural signal may occur simultaneously over the transmitter circuit and second transmitter circuit, respectively. The processing circuit may be configured to rectify and integrate the amplified neural signal, or to detect successive local extrema of the amplified neural signal. The neural signal may be accessed using electrodes in contact with a renal nerve of the living subject. The device may further include a modulator to modulate a periodic waveform using the derivative neural signal. The acquisition circuit and the processing circuit may be housed in a first enclosure, the transmitter circuit may be housed in a second enclosure, and the derivative neural signal may be passed from an output of the first enclosure to an input of the second enclosure. The second enclosure may houses circuitry for monitoring one or more vital signs of an implanted subject, and the input of the second enclosure may be a biopotential input. The processing circuit may be configured to determine and subtract an offset from the derivative neural signal, where the offset represents a contribution of noise to the derivative neural signal.

In a third general aspect, a method of monitoring neural signal activity with an implantable monitoring device implanted in a living subject includes sampling a neural signal, having a signal bandwidth of at least 1000 Hz, at a predetermined sampling frequency less than a Nyquist frequency for the neural signal to produce an undersampled neural signal from which gross activity of the neural signal can be derived. The method also includes wirelessly transmitting the undersampled neural signal from the implantable monitoring device for receipt by a receiver external of the living subject.

The details of one or more implementations are set forth in the accompanying drawings and the description below. Other features, objects, and advantages will be apparent from the description and drawings, and from the claims.

### DESCRIPTION OF DRAWINGS

FIG. 1 is a block diagram of an exemplary implantable device that can be used to acquire, process, and telemeter neural signals.

FIG. 2 is a block diagram of an exemplary system that may be used to acquire, process, transmit, and monitor neural signals originating within an implanted subject.

FIG. 3 is a block diagram of an exemplary device that can be used to acquire, process, and telemeter neural signals.

FIG. 4 is a block diagram of an exemplary implantable device that can be used to acquire, process, and telemeter neural signals, including a raw neural signal and a reduced-bandwidth neural signal.

FIG. 5 is a block diagram of an exemplary single-amplifier implantable device that can be used to acquire a neural signal and provide one or both of the raw neural signal and a lower-bandwidth neural signal representative of the raw neural signal.

Like reference symbols in the various drawings indicate like elements.

### DETAILED DESCRIPTION

This disclosure discusses using an implantable monitoring device to acquire, process, and telemeter neural signals in a power-efficient manner. In some implementations, an implantable monitoring device senses neural signal activity and processes the sensed signal to create a derivative signal of lower bandwidth than the sensed signal, which may be telemetered from the device to an external receiver in a power-efficient fashion, yet which substantially maintains information of interest for a researcher interested in studying neural signal activity of the implanted research subject. In some implementations, the devices, techniques, and systems disclosed herein may be used to monitor sympathetic or parasympathetic neural signal activity. Also, neural signal monitoring may be combined with vital signal monitoring, including a device that monitors neural signal activity in combination with monitoring one or more vital signals, such as temperature, pressure (arterial, venous, endocardial, pressure within a body organ, intra-cranial, etc.), ECG, EMG, EEG, and the like. Various of the device implementations discussed herein may acquire, process, and telemeter the signals in a power-efficient fashion, so as to consume smaller amounts of electrical current, such that battery drain is reduced and battery charge-life is extended. Power efficient operation may be important for chronic-use implantable monitors where miniature size is important, such as for monitoring small animals (e.g., rodents). In some implementations, a small battery may be used to continuously power the implant for weeks or months without recharging.

Power-efficient monitoring of neural signals can be challenging with therapeutic devices, and can also be challenging with monitoring devices used to monitor sympathetic nerve activity in laboratory animals used in biomedical research. In some examples, the full bandwidth of a sympathetic neural signal may be about 1-3 KHz, and a telemetry link to transmit such a signal may consume considerable power. However, in many cases some or all of the high frequency information present in the full bandwidth of the neural signal may not be of interest to the researcher, who may be primarily concerned with gross activity of the neural signal, which gross activity may be preserved by the derivative signal described herein. In some implementations, the derivative signal derived from the sensed sympathetic neural signal may be telemetered from the implantable device to an external receiver outside of the implanted subject using a telemetry channel with a correspondingly reduced bandwidth. This may provide significant power savings, which may lead to increased battery life of the implantable monitoring device, more accurate data collection due to reduced stress of the research subject caused by disruption associated with battery replacement or recharging, and reduced downtime or periods where data may not be collected.

In some implementations, the neural signal bandwidth to be transmitted may be intentionally constrained to reduce current drain and extend battery life. This may improve research results and reduce stress on implanted research subjects, since replacing an expired battery may require surgery, and recharging a depleted battery may involve disruptive handling and/or moving of the subject, in addition to periods where data collection may not be possible. In some implementations, current drain is roughly proportional to number of bits transmitted (for digital transmission) or to the number of pulses transmitted (for pulse-position transmission), each of which may be proportional to signal bandwidth. Some telemetry protocols, such as pulse-position, or telemetry methods, such as low-frequency near-field transmission, may be chosen for their power efficiency but may be limited to transmitting only low-bandwidth signals. In applications where low current drain and long battery life are desired, a choice of these low-bandwidth protocols or methods may be appropriate.

Some implementations of the techniques, devices, and systems described herein can be used to provide neural signal researchers with neural signal information to complete their investigations of nerve recordings, and do so in a manner that results in low power consumption. The techniques, devices, and systems disclosed herein can provide a reduced-bandwidth derivative form of the neural signal that retains the desired information from the nerve recording—that is, gross information that a researcher may consider important in evaluating the neural activity. In some implementations, the sensed neural signal may be rectified and integrated within the implantable device. The lower-bandwidth integrated signal may have similar bandwidth to some physiological parameters that may be impacted by the neural signal, such as time-averaged blood pressure or heart rate. This similarity in respective bandwidths between the various signals may make correlation identification easier or more apparent.

A second mode may be provided that permits the full-bandwidth, raw neural signal to be observed, according to some implementations. This may be useful to test proper placement of sensing electrodes on or near the nerve, and to evaluate system functionality. While this second mode might draw a high amount of current if used continuously, its impact on battery life may be acceptable if used intermittently or when needed.

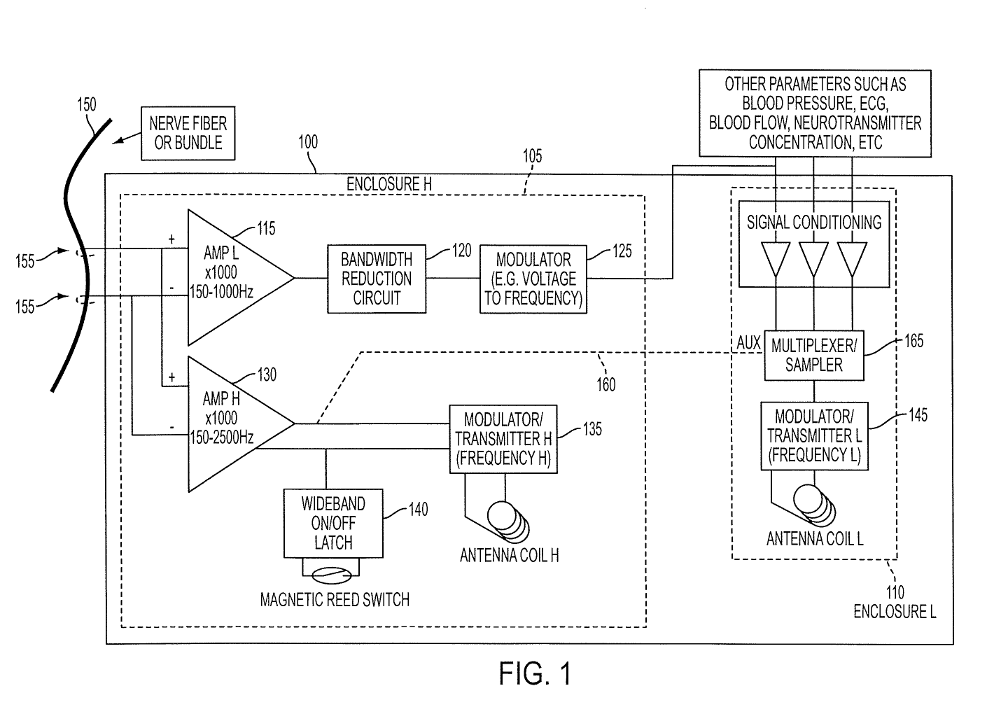

FIG. 1 is a block diagram of an exemplary implementation of an implantable device 100 that can be used to acquire, process, and telemeter neural signals. The implementation shown in FIG. 1 includes two enclosures: a first enclosure 105 labeled “Enclosure H,” and a second enclosure 110 labeled “Enclosure L.” In some implementations, the first enclosure 105 and second enclosure 110 may be combined within a single enclosure (not shown in FIG. 1). Many variations or approaches can be used to implement blocks or portions of the device 100.

The general signal progression in FIG. 1 is from left to right. A first signal path defined by a first operational amplifier 115 (“AMP L”), a bandwidth reduction circuit 120, a modulator 125, and Enclosure L 110 can be used to implement a low-bandwidth transmission portion of the device. A second signal path defined by a second operational amplifier 130 (“AMP H”), and a modulator/transmitter 135 (“Modulator/Transmitter H”) can be used to implement a high-bandwidth transmission portion of the device, and may be used by implementations that include the second mode of operation described above. In some implementations, the high-bandwidth portion may be turned ON and OFF by the user, independent of the low-bandwidth portion. For example, an on/off latch 140 may be used to provide this functionality. Other methods for turning on and off could include sensing an RF field or a magnetic field, for example. In various implementations, the low-bandwidth portion can also be turned ON and OFF (associated hardware not shown in FIG. 1 for simplicity), such that altogether there may be at least three modes of operation (OFF, low-bandwidth only, and low & high bandwidth), or four modes of operation (OFF, low-bandwidth only, high-bandwidth only, and low & high bandwidth). Enclosure L 110 and Enclosure H 105 may be powered independently by separate batteries (not shown in FIG. 1), one in each enclosure, and may be turned ON and OFF independently, according to some implementations.

In some implementations, low-bandwidth transmission and high-bandwidth transmission can occur simultaneously. Transmitter H 135 can be used to transmit the high-bandwidth signal, and Transmitter L 145 (shown within Enclosure L 110 in FIG. 1) can be used to transmit the low-bandwidth signal. The low-bandwidth and high-bandwidth transmissions may occur at different frequencies, such that during the time low- and high-bandwidth signals are concurrently transmitted they are transmitted to respective receivers and are non-interfering. In one implementation, transmitter H 135 transmits at 18 MHz, and transmitter L 145 transmits at 455 KHz. In another implementation, transmitter H 135 transmits at 18 MHz, and transmitter L 145 transmits at 8 MHz.

The circuitry on the right of FIG. 1 is shown within a separate Enclosure L 110 to describe an implementation where Enclosure L 110 is a separate implant. As mentioned, in various implementations, the features described herein may be implemented in single or multiple implanted devices. In some cases, it may be advantageous to use multiple devices to leverage an existing device that can provide some of the functionality described herein. For example, Data Sciences International, of St. Paul, Minn., offers an implantable monitoring device, the C50-PXT, that can be used to sense, process, and telemeter temperature, pressure, and a biopotential in a laboratory animal. In an implementation, the C50-PXT may be used as enclosure L 110. The output of modulator 125 may be received at the biopotential input of the C50-PXT, and telemetered by Transmitter L 145.

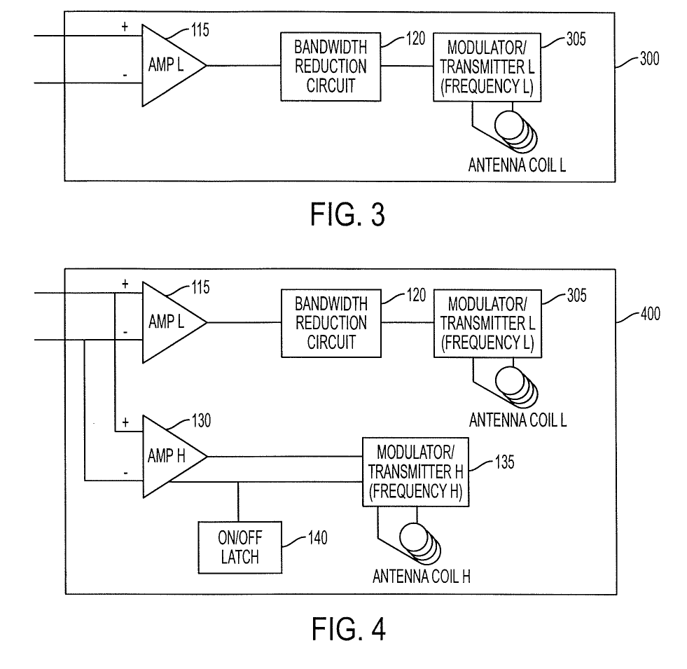

FIG. 3 is a block diagram of an exemplary implantable device 300 that can be used to acquire, process, and telemeter neural signals. Device 300 includes the first operational amplifier 115 (“AMP L”) and the bandwidth reduction circuit 120, described above with reference to FIG. 1, and includes a modulator/transmitter 305 for wirelessly transmitting a signal, such as a reduced-bandwidth neural signal as described above.

FIG. 4 is a block diagram of an exemplary implantable device 400 that can be used to acquire, process, and telemeter neural signals, including a raw neural signal and a reduced-bandwidth neural signal. Device 400 includes the components 115, 120, and 305 from device 300 of FIG. 3, and also includes operational amplifier 130 (“AMP H”), and modulator/transmitter 135 (“Modulator/Transmitter H”), which can be used to implement a high-bandwidth transmission portion of the device, as described above with reference to FIG. 1.

The devices 300 and 400 in FIGS. 3 and 4 use a single enclosure, as opposed to the two-enclosure implementation shown in FIG. 1. In various implementations, device 300 or device 400 may additionally include provisions for acquiring and processing vital signals such as temperature, pressure (arterial, venous, endocardial, pressure within a body organ, intra-cranial, etc.), ECG, EMG, EEG, and the like, which provisions are omitted from the figures for clarity.

Referring again to FIG. 1, neural activity can be sensed from a nerve fiber or bundle 150 with two fine-wire hook connections 155. In one example, the nerve fiber or bundle 150 is the renal nerve of the implanted subject, but any appropriate neural site may be used. Alternative sense connections may be used, such as collagen-based electrodes or other neural electrodes known in the art. A separate common or ground wire (not shown) may connect the tissue 150 and the amplifiers 115, 130 to provide a reference. Exemplary techniques that can be used to access the nerve and stabilize the connections are described in Method for Continuous Measurements of Renal Sympathetic Nerve Activity and Cardiovascular Function During Exercise in Rats, Experimental Physiology, Kenju Miki et al., Vol. 87.1, the contents of which are incorporated by reference herein in their entirety. Many other techniques may be used to access the nerve 150. In some cases, additional electrodes can be used, such as implementations that use quasi-tripolar electrodes. Appropriate modifications to the amplifier blocks 115, 130 may be implemented to facilitate use of these alternative approaches.

The nerve-sense connections sense a differential signal of approximately 100 uV p-p (−50/+1000%) within a frequency band of 150 to 5000 Hz, according to some implementations. The differential amplifiers 115, 130 may have a high common-mode rejection ratio, such that EMG and ECG information that may be present between the common electrode and the nerve electrodes does not corrupt the neural information. The amplifiers 115, 130 may include sufficient gain-bandwidth to amplify the low level neural signals to a level where noise added by subsequent circuits will not be significant. For best results, amplifiers having low input-referred noise may be used so as not to not add significant noise compared to the low (approximately 100 uV p-p) signal level of the sensed neural signal.

In some cases, neural signal sites of interest are near muscle tissue that can generate EMG signals when the muscle expands or contracts. The nearby EMG source may present a differential signal at the nerve-sense connections that can corrupt the neural signal. Configurations such as the quasi-tripolar electrodes noted above may be used to reduce the EMG interference, according to some implementations. Exemplary methods to reduce EMG interference with certain electrode and amplifier configurations are described in Passive Neutralization of Myoelectric Interference from Neural Recording Tripoles, IEEE Transactions on Biomedical Engineering, Vol. 54, No. 6. June 2007, the contents of which are incorporated by reference herein in their entirety.

Remaining EMG interference may be challenging to reject, as its frequency content may typically overlap with that of neural signals. In some implementations, advanced time-frequency analysis or filtering may be used to minimize any corrupting impact of the EMG signal. This filtering may occur prior to creation of the derivative waveform such that the impact of EMG on the derivative waveform is eliminated or minimized. In some cases, a neural signal may present itself in bursts that are synchronous with heart rate. In these cases, windowing or multiplying the neural signal with a signal derived from heart rate or from ECG may be used to reduce EMG or other noise or interference. Any of the processing methods described may be implemented, for example, at the front end of bandwidth reduction circuit 120, and/or between AMP H 130 and modulator/transmitter 135, according to various implementations.

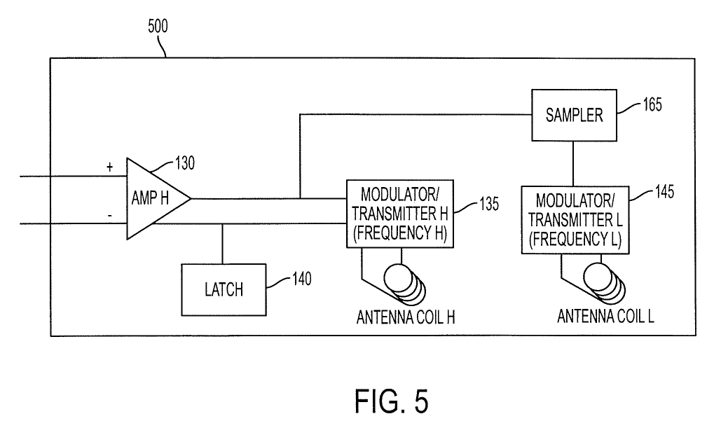

The implementation shown in FIG. 1 includes separate amplifiers for the low- and high-bandwidth signal paths. This approach may reduce current drain, since AMP H 130 may be OFF most of the time, and AMP L 115 may draw lower current than AMP H 130 because it is a lower-bandwidth amplifier. As such, AMP L 115 may be a lower-current amplifier. In other implementations, a single amplifier may be used. Such an approach may be useful, for example, in applications where similar gain-bandwidth requirements are called for. For example, a dotted line 160 from AMP H 130 to AUX represents an alternative, single-amplifier low-bandwidth approach. Using this approach, a high-bandwidth signal can be fed directly to a sampler 165 and sampled at a rate that is lower than the Nyquist sampling rate (i.e., undersampled). In this case, the low-frequency signal path defined by amplifier 115, bandwidth reduction circuit 120, and modulator 125 may be omitted. FIG. 5 is a block diagram of an exemplary single-amplifier implantable device 500 that can be used to acquire a neural signal and provide one or both of the raw neural signal and a lower-bandwidth neural signal representative of the raw neural signal. The device 500 has a single enclosure that houses amp H 130, modulator/transmitter 135, sampler 165 and modulator/transmitter/145, and optionally latch 140.

Referring again to the dual-signal-path implementation shown in FIG. 1, the bandwidth reduction circuit 120 can create a lower-bandwidth derived neural signal, according to an implementation, and can be implemented in several different forms. In some implementations, the bandwidth reduction function can result in a transformation where the input bandwidth is on the order of 150 Hz to 2500 Hz, and the derived output bandwidth is on the order of 0 Hz to 100 Hz. In some cases, for example when neural activity is relatively constant over a period of time, the derived signal may include frequency content near 0 Hz. Because some biopotential channels may not be able to process a 0 Hz signal or a signal with frequency near 0 Hz, modulator 125 can be used to transform a 0 Hz or low-frequency signal to a higher frequency that may be passed by such a biopotential channel. That is, the derivative neural signal can be used to modulate a periodic waveform. In cases where subsequent circuitry is capable of processing 0 Hz or near-0 Hz signals, the modulator 125 may be omitted.

Bandwidth reduction techniques can be applied, for example within bandwidth reduction circuit 120, to an original, amplified neural signal to create a derived signal, and may be implemented by various electronics and signal processing approaches. Bandwidth reduction of the sensed signal may involve a trade-off between preserving all of the information present in the signal, and low power consumption by the processing and telemetering circuitry such that extended battery life may be realized. Some implementations may produce a derived signal that preserves the frequency content or information that is most useful to the research or application being performed, and which does not preserve the frequency content or information that is not useful, and which might therefore cause undue battery current drain if provisions were made to preserve it.

One class of bandwidth reduction technique is known as envelope detection, and may be used to demodulate radio frequency amplitude-modulated signals. This technique tracks the amplitude envelope of the original signal as defined by its peak-to-peak amplitude. For an original signal where the positive and negative excursions from the mean value are similar, peak (or valley) detection may be used as a reasonable surrogate for peak-to-peak detection. Peak (or valley) detection may require less circuitry and battery current than peak-to-peak detection, and therefore may preserve battery life. Two additional parameters related to envelope detection are attack-time (the speed at which increasing amplitude is tracked) and decay-time (the speed at which decreasing amplitude is tracked). The attack-time may typically be inherent to the electrical design and tracks each subsequent peak, but may be slowed in an implementation to limit the bandwidth. The decay-time is normally chosen to track the fastest rate at which the amplitude will decay. In an implementation, the attack-time and decay-time can be chosen or modified by subsequent filtering to optimize the result for the research or application. For example, with slow attack/decay times, the system may be designed to be relatively unresponsive to single or minimal neural action potential firings, but still fully responsive to a burst of activity consisting of numerous firings. As an additional step to reduce battery current consumption, envelope detection can be performed with a non-linear device, such as a bipolar transistor, rather than a higher-current implementation that may use operational amplifiers.

Various alternative techniques can be applied to the original signal from AMP L 115 to accomplish bandwidth reduction. One alternative is to rectify and integrate the original signal, and circuitry may be provided in bandwidth reduction circuit 120 to provide this processing. Another alternative is to include additional nonlinear processing that can accentuate the neural signals and minimize noise, such as by additionally calculating the square of the derived signal. Another alternative includes determining and subtracting an offset from the derived signal, where the offset represents a contribution of noise to the signal. Another alternative is to detect individual neural firings with a threshold detector, filter, or other means, and derive a low-bandwidth signal that corresponds to neural firing rate.

The modulator block 125 converts the low bandwidth signal to an appropriate form to modulate the transmitter 145, according to some implementations. Modulator 125 may be used, for example, when a standard biopotential-input-channel transmitter lacking a 0 Hz (or DC) response is used. To preserve the 0 Hz response, AM or FM modulation can be used to create an appropriate signal for the biopotential input. For example, an “interim” biopotential signal can be generated that can be frequency-modulated from 50 Hz (e.g., to indicate no neural activity) to 100 Hz (e.g., to indicate high neural activity). This FM signal may be decoded by a downstream data acquisition system to recreate the derived neural signal. If a channel with response to 0 Hz is used, the modulator 125 may be bypassed or omitted.

As mentioned above, an alternative to the bandwidth reduction circuit 120 is to under-sample the high-bandwidth neural signal. This approach is represented by the dotted line 160 from AMP H 130 to AUX in FIG. 1. Using this approach, AMP L 115, bandwidth reduction circuit 120, and modulator 125 may be omitted. As used herein, undersampling refers to sampling the signal at a lower sample rate than would satisfy the Nyquist criteria. The Nyquist criteria or Nyquist rate refers to a sample rate of twice the highest-frequency component of the signal. Sampling at the Nyquist rate or at a higher sample rate preserves all of the frequency content of the signal.

In cases where a researcher is interested in the general activity of the signal and not the shape or morphology of the signal, as is frequently the case for researchers interested in monitoring neural activity in laboratory research animals, it may be acceptable to lose high-frequency content of an acquired neural signal. For example, if a 150-2500 Hz neural signal is sampled at 500 Hz, and later low-pass filtered to 150 Hz, the frequency content at the sample rate and its harmonics would be mixed down to “baseband.” That is, the frequency content that was at 350-650 Hz (i.e., first harmonic information: 500 Hz+−150 Hz), 850-1150 Hz (i.e., second harmonic information: 1000 Hz+−150 Hz), 1350-1650 Hz (i.e., third harmonic information: 1500 Hz+−150 Hz), and so on, would appear in the derived signal at 0-150 Hz. This derived (0-150 Hz) signal can be transmitted via a standard transmitter (including, for example, by the transmitter in the C50-PXT, offered by Data Sciences International of St. Paul, Minn.) as a biopotential signal with bandwidth of 1-150 Hz. The transmitted signal may be received by an external receiver, such as a receiver included in an external monitoring unit. The received signal may be processed by hardware or software within or outside of the external monitoring unit, according to an implementation. In one example, software running on a computing system rectifies the received signal and performs a moving average to provide a useful signal to a researcher, as by displaying information related to the neural signals on a display screen. The researcher may use this information, for example, to assess an administered drug's effect on the research subject, which may provide useful information for biomedical research.

In some implementations, the techniques, devices, and systems described herein can be used to create a derivative waveform with a bandwidth of less than 100 Hz from an acquired neural signal having a bandwidth in excess of about 1000 Hz within an implantable monitoring device. For example, the bandwidth reduction circuit 120 may create such a derivative waveform. The derivative waveform may represent the magnitude of the nerve activity. In other implementations, the techniques, devices, and systems described herein can be used to sample a nerve signal at less than the associated Nyquist rate in a manner that preserves information in the signal relating to the magnitude of the nerve activity. This may be referred to as power-conservation sampling (PCS).

Either the derivative signal or the PCS-derived signal may be suitable for measurement by a biopotential input channel, such as an ECG input channel, of an implantable telemetry device. Such an implantable telemetry device may also be capable of measuring blood pressure and other vital signals.

Using the devices, techniques, and systems discussed herein, separate transmitter circuits may be used to simultaneously telemeter a high bandwidth nerve signal and a lower-bandwidth derivative waveform that represents the magnitude of the nerve signal. This mode of simultaneous transmission may be used to check and validate system performance and function. Simultaneous (or alternatively, independent) display of high bandwidth nerve recordings and the derivative waveform that represents the magnitude of the nerve signal may be provided on a display device.

A nerve signal measurement module (NSMM) may be provided that combines either the derivative waveform means or the PCS means described above to produce a signal with a bandwidth of less than 250 Hz that is representative of the magnitude of nerve activity. The circuit for telemetering the high bandwidth raw signal may be also contained in the module and the high bandwidth telemetering circuit may be remotely switched on and off by a magnet or wireless communication means, according to some implementations, and it may be able to automatically activate the high bandwidth telemetry mode at preprogrammed times. The input of the module may be connected to electrodes suitable for sensing the electrical activity of the nerve, and the output of the module may be suitable for connecting to the ECG input channel of an implantable telemetry device. The implantable telemetry device may also be capable of monitoring blood pressure and possibly other signals from within the body.

The NSMM may be connected to the biopotential input channel of an implantable telemetry device or may be incorporated within the housing of an implantable telemetering device.

Pathophysiology research areas that may benefit from chronic recording of neural signals, or from concurrent chronic recording of neural signals and other vital signals (e.g., pressure, ECG, etc.) include hypertension, diabetes and associated neuropathy, arrhythmia (ventricular or atrial), heart failure, and sleep apnea, to list just a few examples. Data provided by the techniques, devices, and systems disclosed herein may be useful not only for understanding mechanisms of disease, but also for developing therapy.

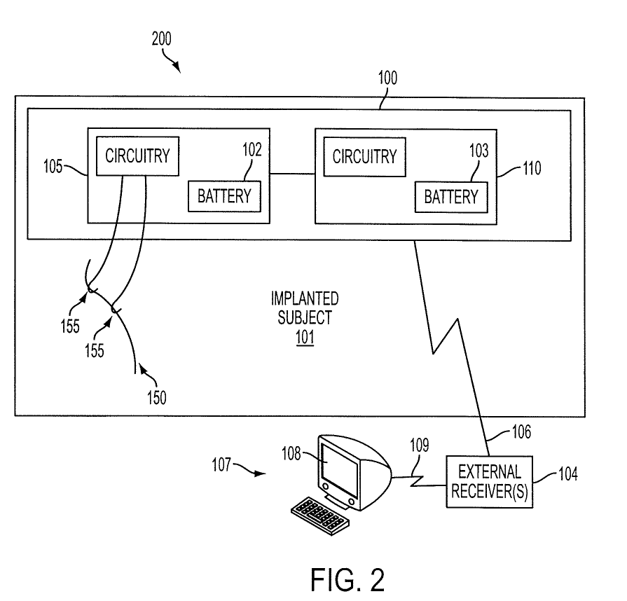

FIG. 2 is a block diagram of an exemplary system 200 that may be used to acquire, process, transmit, and monitor neural signals originating within an implanted subject. Implantable device 100 is shown implanted in an implanted subject 101. Examples of implanted subjects 101 may include mice, rats, primates, dogs, or any other appropriate laboratory animal. In some implementations, implanted device 100 may be implanted in a human being, and may be used to monitor neural signals originating within the human. In some implementations, a human patient may be further implanted with a therapy device, such as a pacemaker, defibrillator, drug pump, vascular access pump, or the like, and neural signal information from implantable device 100 may be used to adjust a therapy to the patient. For example, if neural signal information provided by device 100 indicates a likelihood that an associated physiological change may occur soon or in the future, a therapy to the patient may be initiated or adjusted in response. In some cases, the adverse physiological change may be prevented as a result, or adverse effects of the physiological change may be minimized. In yet other implementations, device 100 may include a therapy component, such as any of those mentioned above, and may be capable of issuing a therapy to the patient.

As described above, depending upon the implementation, the device 100 may include a single enclosure or multiple enclosures. The FIG. 2 implementation shows two enclosures 105, 110, each of which contains a battery (102 and 103, respectively). Blocks labeled “circuitry” within the enclosures 105, 110 may include the circuitry shown in FIG. 1 for the respective enclosure, details of which are not repeated in FIG. 2 for simplicity. Additional circuitry such as one or more microprocessors or microcontrollers, memory, control circuitry, etc., may also be included, but is not shown for simplicity.

The implanted device 100 may communicate with one or more external receivers 104 over one or more communication channels 106. Communication channel(s) 106 may include wireless communications as described above. In some implementations, device 100 communicates with a first receiver over a first communication channel for low-bandwidth signal communication, and communicates with a second receiver over a second communication channel for higher-bandwidth signal communication. In some implementations, the two communication channels 106 may operate simultaneously, such that the device 100 is transmitting both lower-bandwidth neural information and higher-bandwidth neural information concurrently to one or more external receivers 104. The one or more receivers 104 may be housed, for example, within a base monitoring station (not shown in FIG. 2). The base monitoring station may transmit the information received from the one or more receivers 104 over a communication path 109 to a system 107 that includes a display screen 108 where information may be displayed to a user, such as a physician or medical care provider. In some implementations, the base station and the system 107 may be the same unit. In some implementations, the system 107 may comprise a computing system, such as a personal computer, a server, or the like. Software running on the base station or system 107 may process the received information in preparation for displaying the information on the display screen 108.

Using the techniques disclosed herein, the batteries 102, 103 (or in the case of a single-enclosure device, a single battery) of the implanted device 100 may provide longer life due to reduced current consumption of the associated circuitry.

In various implementations, the derivative signal and/or the undersampled signal may be suitable for measurement by a biopotential input channel of an implantable telemetry device. The biopotential input channel may be an ECG input channel. The implantable telemetry device may be capable of measuring blood pressure and other vital signals. The implantable monitoring device may include circuitry for sampling a high-bandwidth nerve signal, and the sampled high-bandwidth nerve signal and the derivative waveform may be simultaneously telemetered from the implantable monitoring device. A system that simultaneously displays the high-bandwidth nerve signal and the derivative waveform may be used to check and validate performance of the implantable monitoring device and the system.

In an implementation, a nerve signal measurement module (NSMM) senses a high-bandwidth neural signal and combines either the means for producing the derivative waveform or the undersampled waveform to produce a signal with a bandwidth of less than 250 Hz that is representative of the magnitude of nerve activity. A circuit for telemetering the high bandwidth neural signal can be contained in the module, and may be remotely switched on or off by a magnet or wireless communication means. The high-bandwidth telemetry circuit may be automatically activated at preprogrammed times, according to some implementations. An input of the module may be connected to electrodes suitable for sensing the neural electrical activity. In various implementations, the output of the module may be suitable for connecting to a biopotential input of an implantable telemetry device. The biopotential input may be an ECG input channel. The implantable telemetry device may be capable of monitoring blood pressure or other signals from within the body. The NSSM may be connected to the biopotential input channel of an implantable telemetry device or may be incorporated within the housing of an implantable telemetering device.

A number of implementations have been described. Nevertheless, it will be understood that various modifications may be made without departing from the spirit and scope of the techniques, devices, and systems disclosed herein. For example, any of the components or circuitry from Enclosure L 10 in FIG. 1 may be included in Enclosure H 105, or vice versa. Also, it will be understood that the energy providing means for the implantable device described herein as a battery may be replaced by other energy providing means, and that advantages provided by the techniques, devices, and systems disclosed herein may be similarly beneficial in extending the useful life of the device regardless of the energy-providing means used. When a battery is used, it may be a rechargeable battery, where the interval between charging the rechargeable battery may be extended by use of the battery current saving techniques described. Energy sources other than a primary battery may be utilized, possibly in conjunction with a rechargeable battery or a capacitor for energy storage. Examples of such energy sources include electromagnetic energy that may be harvested by a coil, antenna, or sensor in the implant, energy from motion or flexure of the subject that may be harvested by a piezoelectric or other material, chemical energy from substances within the body of the subject, or thermal energy. Accordingly, other embodiments are within the scope of the following claims.

What is claimed is:

1. A method of monitoring neural signal activity with an implantable monitoring device implanted in a living subject, comprising:
amplifying a neural signal having a signal bandwidth of at least 1000 Hz;
processing the amplified neural signal to produce a reduced-bandwidth derivative neural signal representative of gross activity of the neural signal; and
wirelessly transmitting the derivative neural signal from the implantable monitoring device for receipt by a receiver external of the living subject.

2. The method of claim 1, wherein the derivative neural signal has a signal bandwidth of less than about 250 Hz.

3. The method of claim 1, further comprising sampling the amplified neural signal and wirelessly transmitting the sampled signal for receipt by a receiver external of the living subject, wherein the sampling preserves full bandwidth of the amplified neural signal.

4. The method of claim 3, wherein transmission of the derivative neural signal and the full-bandwidth sampled signal occurs simultaneously.

5. The method of claim 1, wherein processing the amplified neural signal includes rectifying and integrating the amplified neural signal.

6. The method of claim 1, wherein processing the amplified neural signal includes detecting successive local extrema of the amplified neural signal.

7. The method of claim 1, further comprising determining and subtracting an offset from the derivative neural signal, where the offset represents a contribution of noise to the derivative neural signal.

8. The method of claim 1, further comprising using the derivative neural signal to modulate a periodic waveform.

9. The method of claim 1, wherein the neural signal is accessed using electrodes in contact with a renal nerve of the living subject.

10. The method of claim 1, further comprising assessing the derivative neural signal to predict occurrence of a future physiological change for the living subject.

11. An implantable monitoring device, comprising:
an acquisition circuit configured to amplify a neural signal having a signal bandwidth of at least 1000 Hz;
a processing circuit configured to process the amplified neural signal to produce a derivative neural signal representative of gross activity of the neural signal; and
a transmitter circuit configured to wirelessly transmit the derivative neural signal from the implantable monitoring device for receipt by a receiver external of the living subject.

12. The implantable monitoring device of claim 11, further comprising a control circuit that when activated causes the transmitter circuit to wirelessly transmit the amplified neural signal for receipt by a receiver external of the living subject.

13. The implantable monitoring device of claim 12, further comprising a second transmitter circuit, and wherein transmission of the derivative neural signal and the amplified neural signal occurs simultaneously over the transmitter circuit and second transmitter circuit, respectively.

14. The implantable monitoring device of claim 11, wherein the processing circuit is configured to rectify and integrate the amplified neural signal.

15. The implantable monitoring device of claim 11, wherein the processing circuit is configured to detect successive local extrema of the amplified neural signal.

16. The implantable monitoring device of claim 11, wherein the processing circuit is configured to determine and subtract an offset from the derivative neural signal, where the offset represents a contribution of noise to the derivative neural signal.

17. The implantable monitoring device of claim 11, further comprising a modulator to modulate a periodic waveform using the derivative neural signal.

18. The implantable monitoring device of claim 11, wherein the neural signal is accessed using electrodes in contact with a renal nerve of the living subject.

19. The implantable monitoring device of claim 11, wherein the acquisition circuit and the processing circuit are housed in a first enclosure, the transmitter circuit is housed in a second enclosure, and wherein the derivative neural signal is passed from an output of the first enclosure to an input of the second enclosure.

20. The implantable monitoring device of claim 19, wherein the second enclosure houses circuitry for monitoring one or more vital signs of an implanted subject, and wherein the input of the second enclosure is a biopotential input.

21. A method of monitoring neural signal activity with an implantable monitoring device implanted in a living subject, comprising:
sampling a neural signal, having a signal bandwidth of at least 1000 Hz, at a predetermined sampling frequency less than a Nyquist frequency for the neural signal to produce an undersampled neural signal from which gross activity of the neural signal can be derived; and
wirelessly transmitting the undersampled neural signal from the implantable monitoring device for receipt by a receiver external of the living subject.
